(19) United States
(12) Patent Application Publication
(10) Pub. No.: US 2020/0022599 A1
(43) Pub. Date: Jan. 23, 2020

(54) ENERGY-SAVING SWITCHING METHOD AND A SMART WATCH WITH HEART RATE DETECTION FUNCTION

(71) Applicant: SHANGHAI ZHANGMEN SCIENCE AND TECHNOLOGY CO., LTD., Shanghai (CN)

(72) Inventors: Chunda YIN, Shanghai (CN); Peng Zhao, Shanghai (CN)

(21) Appl. No.: 16/587,036

(22) Filed: Sep. 29, 2019

Related U.S. Application Data
(63) Continuation of application No. PCT/CN2017/119842, filed on Dec. 29, 2017.

(30) Foreign Application Priority Data
Mar. 31, 2017 (CN) 201710207500.6

(51) Int. Cl. A61B 5/024; A61B 5/00

(52) U.S. Cl. CPC A61B 5/02438; A61B 5/681; A61B 5/002; A61B 2560/0209; A61B 5/6844; A61B 5/7455; A61B 5/746

(57) ABSTRACT

A method and a smart watch for detecting and prompting abnormal heart rate data, and the start watch comprises a main processor and an auxiliary processor. When the main processor is in a dormancy state, the auxiliary processor collects heart rate data and analyzes whether the heart rate data is abnormal. When it is determined that the heart rate data is abnormal, the auxiliary processor is interrupted and the main processor is woken up, and the abnormal data is submitted to the main processor for processing. When the processing is completed, the main processor returns to the dormancy state, so that the main processor does not need to participate in the heart rate data collection and analysis process. The power consumption during running of the auxiliary processor is one quarter of the power consumption of the main processor in the dormancy state.

### PRIORITY CLAIM

This application is the Continuation Application of International Application No. PCT/CN2017/119842, filed on Dec. 29, 2017, which claims priority to Chinese Patent Application No. 201710207500.6, filed on Mar. 31, 2017, both of which are incorporated by reference in their entireties for any and all purposes.

### FIELD

The present disclosure relates to a technical field of smart watch, and particularly relates to a method and a smart watch for detecting and prompting abnormal heart rate data.

### BACKGROUND

As an important physiological indicator of blood circulation function, heart rate is not only an important indicator reflecting the strength of heart function, but also reflects the intensity of human exercise and thus is also widely used in daily exercise. It can be used as an index and basis for assessing the appropriateness of exercise load, as well as the state of cardiac function, and as an indicator for determining whether the body has excessive fatigue and assessing the degree of exercise in a certain stage. In the prior art, smart wearable products mainly based on smart watches have begun to widely incorporate the heart rate detection function, which brings great convenience to self-test for the users. However, the existing heart rate wristband or heart rate watch collects heart rate mainly by using a traditional LED photoelectric heart rate collection method. The power consumption of this method is relatively high due to the existence of LED, for example about 10-20 mA. In order to extend the use time, it can only be turned on when the measurement is being taken, and it has to be turned off at other times. This affects the continuity of heart rate measurement, which in turn affects the quality of health monitoring, thus greatly limiting the wide application of heart rate detection.

Therefore, it is a very urgent need to use the heart rate detection more conveniently while not greatly consuming the power of the smart watch.

### CONTENTS OF THE DISCLOSURE

In view of the above, an object of the present disclosure is to provide a method and a smart watch for detecting and prompting abnormal heart rate data, which can detect and prompt abnormal heart rate in time, thereby satisfying people's daily health detection needs to the most.

According to an aspect of the present disclosure, a method for detecting and prompting abnormal heart rate data is provided. The method comprises:

collecting detected heart rate data;

analyzing the collected heart rate data by an auxiliary processor;

determining whether the collected data is in a threshold range;

triggering to wake up a main processor and the main processor switching from a dormancy state to an operation state when the auxiliary processor detects that the collected heart rate data is abnormal for over a predetermined time period;

establishing communication with a mobile terminal via Bluetooth and transmitting abnormal heart rate and providing an alert prompt by the main processor.

In one embodiment, after determining whether the collected data is in the threshold range, the method comprises: detecting the duration of the abnormal heart rate data; if the duration of the abnormal heart rate data exceeds a predetermined time period, determining the wearing condition of a watch; if the watch is being worn, determining whether a user of the watch is in exercise; if the user is not in exercise, interrupting the auxiliary processor in a low power state and triggering to wake up the main processor to establish connection with the mobile terminal via Bluetooth to provide an alert prompt.

In one embodiment, after the main processor switching from the dormancy state to the operation state, the method further comprises:

connecting to a mobile phone terminal of the user via Bluetooth by the main processor;

transmitting the abnormal heart rate data to the mobile phone terminal of the user by the main processor;

providing an abnormal heart rate prompt by the mobile phone terminal;

wherein the prompt provided by the mobile phone terminal includes any of mobile phone vibration, ringtones, SMS, custom voice.

In one embodiment, the main processor is in the dormancy state and the auxiliary processor is in the low power state when collecting detected heart rate data.

In one embodiment, determining the wearing condition of the watch comprises: turning on an infrared distance detection sensor to detect whether there is an obstruction; obtaining collected heart rate data when obstruction is detected and comparing the collected heart rate data to a predetermined range of wrist heart rate data of normal people; determining that the obstruction is the wrist of the user when the heart rate data is normal, otherwise the obstruction is other obstructions, thus determining whether the watch is being worn by the user.

According to another aspect of the present disclosure, a smart watch is also provided. The smart watch comprises: a main processor, an auxiliary processor, a system service bus, a memory, a heart rate collection processing module, an acceleration collection processing module, and a Bluetooth module, wherein the main processor, the auxiliary processor, and the memory communicate with one another via the system service bus respectively, and the heart rate collection processing module and the acceleration collection processing module are connected to the auxiliary processor.

In one embodiment, the main processor is in a dormancy state and the auxiliary processor is in an operation state when the heart rate data is being collected and analyzed.

In one embodiment, when the heart rate data obtained by the auxiliary processor is abnormal for over a predetermined time period, the main processor is woken up.

In one embodiment, the main processors provides an alert indication after receiving the abnormal data, wherein the alert indication comprises vibrating the smart watch for a time period and transmitting the abnormal data to an intelligent mobile terminal.

In one embodiment, the main processor enters into the dormancy state after transmitting the abnormal data to the mobile terminal.

The beneficial effects of the disclosure lie in: the main processor of the smart watch does not participate in the heart rate data collection and processing in most cases, so that the main system of the smart watch can remain in the dormancy state; the auxiliary processor chip itself has extremely low power consumption, which is equivalent to one quarter of the power consumption of the main processor of the smart watch in dormancy state, thereby saving system power consumption. In addition, when the detected heart rate data is abnormal for over a predetermined period of time, the main processor is woken up, and an alert prompt is transmitted to the mobile phone terminal, so that the abnormal heart rate condition of the user is detected and alerted in time.

### DESCRIPTIONS OF THE DRAWINGS

The drawings are only for the purpose of illustrating the embodiments and are not considered as a limitation of the disclosure. Throughout the drawings, the same reference numerals are used to refer to the same parts. In the drawings:

FIG. 1 shows a structural block diagram of an energy-saving watch in prior art;

FIG. 2 shows a structural block diagram of the smart watch according to an embodiment of the present disclosure;

FIG. 3 shows a flowchart of a method for detecting abnormal heart rate data by the smart watch according to an embodiment of the present disclosure.

### DETAILED EMBODIMENTS

Exemplary embodiments of the present disclosure will be described in more detail below with reference to the accompanying drawings. While the exemplary embodiments of the present disclosure are shown in the drawings, it is understood that the disclosure may be embodied in various forms and not limited by the embodiments set forth herein. Rather, these embodiments are provided so that this disclosure will be more fully understood.

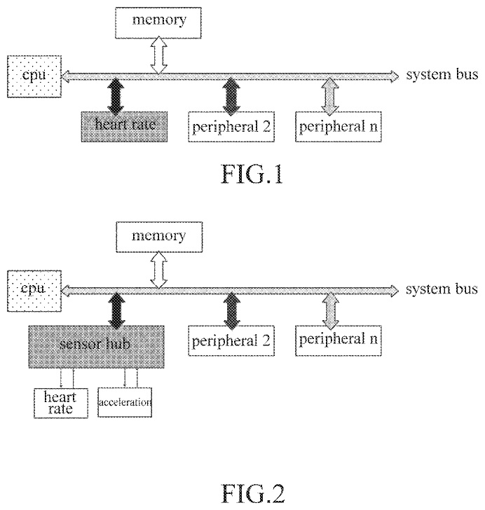

As shown in FIG. 1, a smart watch structure frame of prior art is shown, in which a heart rate measurement module communicates directly with a CPU via a system bus, and the CPU is always in an operating state.

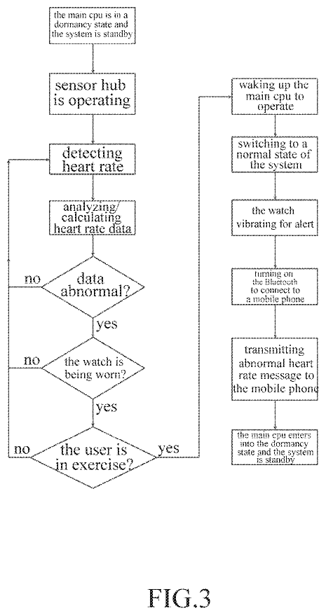

According to an aspect of the present disclosure, an energy-saving smart watch with heart rate detection is provided. As shown in FIG. 2, when the smart watch is in operating state, it can intuitively display information and switch among other application functions. When the smart watch is in dormancy or standby state, the system is in a low power mode. In the low-power mode, the smart watch only displays the time and monitors the heart rate of the user. The ECG collection processing module interacts with the system bus via the auxiliary processor module, such as a sensor hub.

The watch comprises: a watch case, a main processor and a system service bus arranged in the watch case, wherein the main process is respectively connected to a display module, an auxiliary processor module, a wireless communication module and a storage module, and peripherals such as a battery module via the system service bus. The above mentioned modules respectively implement data exchange with the system service bus via a bus such as i2c, spi, usb, etc. The auxiliary processor module communicates with an ECG collection processing module and an acceleration collection processing module, and feeds the obtained data to the main processing.

In one embodiment, the peripherals also include a three-axis gyroscope, a gravity sensor, a navigation component, etc.

In one embodiment, the main processor is an ARM Cortex-M3 as a controller EFM32G200, and the wireless communication module uses a CC2540 chip.

In one embodiment, the acceleration collection module comprises a three-axis acceleration sensor, and the three-axis acceleration sensor comprises a chip LIS3DH for sensing the acceleration change of the device, thereby recognizing the feasibility of a gesture action.

In one embodiment, the battery module is powered by a lithium battery. The 5V voltage output by the MiniUSB is directly supplied to the lithium battery charging management chip TP4056, which is used for managing the charging of the lithium battery. The chip is a complete single-cell lithium ion battery with a constant current, constant voltage linear charger, up to 1A charging current. When the input voltage is removed, TP4056 automatically enters a low current state, reducing battery leakage current to lower than 2 uA.

In one embodiment, the auxiliary processor module is a chip, and the model may be an MCU chip of MSP430F5524IRGCT, which communicates data with the main processor via a data bus.

In one embodiment, the storage module uses a CC2540 ROM, and the ROM value is sufficient for storing the Bluetooth protocol stack, the collected data and the instruction code. The chip does not need an external memory.

In one embodiment, when the smart watch is in a dormancy state, the auxiliary processor module communicates with the ECG collection processing module, and the ECG collection processing module performs arithmetic processing on the collected data.

The ECG collection module performs arithmetic processing on the collected peripheral data, including:

One. Pre-processing. First, the collected signal needs to be smoothed and filtered.

Two. Peak detection. In the continuous signal, the peak is found by locating the value with reciprocal of 0. In the sampled discrete signal, the peak is found by differential operation. After a simple 5-point smoothing filtering method is used in step one, the small error of the sampled data is removed. In order to eliminate the interference, 70% of the R peak amplitude is used as a threshold, and values less than this threshold is set to 0. Through the above coarse screening, possible peaks are left.

Three, DC and AC component extraction. The peak detected in step two divides the PPG signal into several small peaks, called the beat cycle. The DC component is estimated by using the mean of the beat cycle while finding the peak of the beat cycle, and the AC component is obtained from the difference between the peak and the valley of the value.

Four, PPG signal determination. According to the position, amplitude, interval and other information of the peaks and valleys obtained in the previous two steps, it is determined whether this signal is a valid PPG signal.

Five, heart rate calculation:

an average time of the RR interval in a time period is required to calculate the heart rate.

The equation for calculating the average time of the RR interval is: RR interval=(the position of the last R peak−the position of the first R peak)/(360*(the number of the R wave−1));

The equation for calculating the heart rate is: HR=60/RR interval; HR=60*360*(the number of R wave−1)/(the position of the last R peak−the position of the first R peak),

wherein the position of the R peak refers to which data point it is in the ECG signal, and 360 is the sampling frequency of the ECG data.

In one embodiment, the auxiliary processor module implements information exchange with the ECG collection processing module and the acceleration collection processing module via i2C or other data bus.

In one embodiment, the smart watch can launch or exit corresponding function by the user's active operation.

When the main processor of the smart watch is in a dormancy state and the auxiliary processor is kept in a low-power state, the data sent back to the auxiliary processor by the peripherals is analyzed for calculation. When the heart rate data obtained by the auxiliary processor is abnormal for over a predetermined time period, the main processor is woken up and the main processor switches from the dormancy state to an operation state and the auxiliary processor switches from the low power state to an interruption state. The main processor transmits the abnormal heart rate data to a mobile phone terminal via the Bluetooth module. The main processor switches from the operation state to the dormancy state and the auxiliary processor switches from the interruption state to the low power state after transmitting to the mobile phone terminal.

According to an embodiment of the present disclosure, a method for detecting and prompting abnormal heart rate data is provided. As shown in FIG. 3, the auxiliary processor is kept in the low power state when the main processor is in the dormancy state. The auxiliary processor detects and analyzes the data from all the peripherals and submits key data or abnormal data to the main processor as necessary to wake up the main processor for processing.

Step 100: the main processor is in the dormancy state.

Step 101: the sensor hub as the auxiliary processor is kept in an operation state.

Step 102: collecting detected heart rate data.

In one embodiment, the user can set different threshold ranges of heart rate for different ambient scenarios to represent the heart rate threshold of human in different scenarios. As an example, it can set such that the threshold range of heart rate of the user is 50 to 70 in sleep state, the threshold range of heart rate of the user is 80 to 150 in exercise state, and the threshold range of heart rate of the user is 65 to 90 in static scenario. The interval of heart rate detection can also be set, or heart rate detection can be real time. As an example, it can be set that heart rate detection is performed once every 5 minutes in exercise scenario, heart rate detection is performed once every 30 minutes in static scenario, and heart rate detection is performed once every 10 minutes in sleep scenario. Further, heart rate collection can be set as real-time mode.

Step 103: analyzing and calculating the collected heart rate data.

In one embodiment, when there is no historic data, it is determined whether the heart rate data is abnormal according to a range of heart rate of normal people. When there is historic data, the heart rate data is compared with the historic data and it is determined whether the change of the collected heart rate data exceeds a threshold. If yes, abnormal heart rate data is submitted, otherwise heart rate data collection is continued.

Step 104: determining whether the collected data is in a threshold range.

Step 105: if yes, proceeding to step 106, otherwise returning to step 102.

Step 103: determining the wearing condition of the watch. If the watch is being worn by the user, proceeding to step 107, otherwise returning to step 102.

In one embodiment, an infrared distance detection sensor is turned on to detect whether there is an obstruction. When obstruction is detected, the collected heart rate data is obtained and is compared with a predetermined range of wrist heart rate data of normal people. When the heart rate data is normal, it is determined that the obstruction is the wrist of the user, otherwise the obstruction is other obstructions.

In one embodiment, when analyzing the abnormal heart rate data, it is determined whether the monitored heart rate is less than a preset minimum threshold. When the heart rate is detected to be less than the minimum threshold, it is indicated that the smart watch is being worn by no one and the detected data is not used as abnormal heart rate data.

Step 107: determining whether the user is in exercise, and if no, proceeding to step 108, otherwise returning to step 102.

In one embodiment, according to the different ambient threshold ranges set in step 102, it is determined whether the heart rate value mutation occurs when the user is in exercise state. The heart rate data is not treated as abnormal data if the user is in exercise scenario. The heart rate data is treated as abnormal data if the user is not in exercise state.

In one embodiment, when the user is in exercise state, an instruction of waking up the main processor is issued when the heart rate of the user exceeds the maximum threshold of the range of heart rate in exercise state.

Step 108: triggering to wake up the main processor and analyzing the abnormal heart rate data.

In one embodiment, the auxiliary processor issues a wake up instruction to the main processer when it is analyzed that the heart rate data is abnormal.

In one embodiment, the heart rate data is determined directly as abnormal data when the detected heart rate of the user is over 180 times/minute and an emergency instruction is issued to the main processor regardless of the state of the user.

Step 109: the main processor switching from the dormancy state to the operation state.

The main processor issues an instruction and the smart watch switches from the dormancy state to the operation state when it is analyzed that the heart rate data is abnormal data.

Step 110: the watch vibrating to provide abnormal heart rate prompt.

In one embodiment, the watch vibrates for 5 seconds to prompt the user of the abnormal heart rate condition. Different vibration time periods can be set according to the type of the alert or prompt.

Step 111: the main processor connecting to the mobile phone of the user via wireless communication.

In one embodiment, the main processor establishes communication with the mobile phone via Bluetooth.

In one embodiment, the main processor communicates with the mobile phone in UART transmission mode by using a serial port to output and input data.

Step 112: transmitting the abnormal heart rate data to the mobile phone terminal of the user and providing an alert prompt.

In one embodiment, data is transmitted to the mobile phone terminal via Bluetooth or WIFI. The wireless communication module is mainly a wireless transmission module. It uses CC2540 of TI Corporation and implements data communication with microcontrollers using a standard UART port. It transmits data to a mobile phone, such as an emergency contact of the user to ask help from the emergency contact, thus timely rescue and treatment for the user can be achieved. In addition, the data obtained can be analyzed for the second time at the mobile phone terminal to determine the change of the heart rate data.

In one embodiment, the alert prompt includes any of mobile phone vibration, ringtones, SMS, custom voice.

Step 113: the main processor of the smart watch entering into the dormancy state after transmitting the abnormal heart rate data to the mobile phone terminal.

What is claimed is:

1. A method for detecting and prompting abnormal heart rate data, the method comprising:
collecting detected heart rate data;
analyzing the collected heart rate data by an auxiliary processor;
determining whether the collected data is in a threshold range;
triggering to wake up a main processor and the main processor switching from a dormancy state to an operation state when the auxiliary processor detects that the collected heart rate data is abnormal for over a predetermined time period;
establishing communication with a mobile terminal via Bluetooth and transmitting abnormal heart rate and providing an alert prompt by the main processor;
wherein after determining whether the collected data is in the threshold range, detecting the duration of the abnormal heart rate data; if the duration of the abnormal heart rate data exceeds a predetermined time period, determining wearing condition of a watch; if the watch is being worn by a user, determining whether the user is in exercise; if the user is not in exercise, interrupting the auxiliary processor in a low power state and triggering to wake up the main processor to establish connection with the mobile terminal via Bluetooth to transmit abnormal data and provide the alert prompt.

2. The method of claim 1, wherein after the main processor switches from the dormancy state to the operation state, the main processor the main processor connects to a mobile phone terminal of the user via Bluetooth by the main processor, transmits the abnormal heart rate data to the mobile phone terminal of the user by the main processor, and provides an abnormal heart rate prompt by the mobile phone terminal;
wherein the prompt provided by the mobile phone terminal includes any of mobile phone vibration, ringtones, SMS, custom voice.

3. The method of claim 2, wherein after the main processor transmitting the abnormal heart rate to the mobile phone terminal of the user, the main processor switching to the dormancy state.

4. The method of claim 1, wherein the main processor is in the dormancy state and the auxiliary processor is in a lower power state when the heart rate data is being collected.

5. The method of claim 1, wherein determining the wearing condition of the watch comprises turning on an infrared distance detection sensor to detect whether there is an obstruction; determining that the watch is not being worn when no obstruction is detected; obtaining collected heart rate data when obstruction is detected and comparing the collected heart rate data to a predetermined range of wrist heart rate data of normal people; determining that the obstruction is the wrist of the user and the watch is being worn by the user when the heart rate data is normal, otherwise the obstruction is other obstructions and the watch is not being worn by the user.

6. A smart watch comprising:
a main processor, an auxiliary processor, a system service bus, a memory, a heart rate collection processing module, an acceleration collection processing module, and a Bluetooth module, wherein the main processor, the auxiliary processor, and the memory communicate with one another via the system service bus respectively, and the heart rate collection processing module and the acceleration collection processing module are connected to the auxiliary processor, wherein the smart watch
collects detected heart rate data;
analyzes the collected heart rate data by the auxiliary processor;
determines whether the collected data is in a threshold range;
triggers to wake up the main processor and the main processor switching from a dormancy state to an operation state when the auxiliary processor detects that the collected heart rate data is abnormal for over a predetermined time period;
establishes communication with a mobile terminal via Bluetooth and transmitting abnormal heart rate and providing an alert prompt by the main processor;
wherein after whether the collected data is in the threshold range is determined, the duration of the abnormal heart rate data is detected; if the duration of the abnormal heart rate data exceeds a predetermined time period, wearing condition of a watch is determined; if the watch is being worn by a user, whether the user is in exercise is determined; if the user is not in exercise, the auxiliary processor in a low power state is interrupted and the main processor is triggered to be waked up to establish connection with the mobile terminal via Bluetooth to transmit abnormal data and provide the alert prompt.

7. The smart watch of claim 6, wherein the main processor is in the dormancy state and the auxiliary processor is in a lower power state when the heart rate data is being collected.

8. The smart watch of claim 6, wherein waking up the main processor when the heart rate data obtained by the auxiliary processor is abnormal for over a time period.

9. The smart watch of claim 8, wherein after receiving the abnormal data, establishing connection with the Bluetooth module and providing an alert indication by the main processor, and the alert indication comprises vibrating the smart watch for a time period and transmitting the abnormal data to an intelligent mobile terminal.

10. The smart watch of claim 9, wherein the main processor switching to the dormancy state after transmitting the abnormal heart rate data to the mobile terminal.
